(19) United States
(12) Patent Application Publication
(10) Pub. No.: US 2005/0233941 A1
(43) Pub. Date: Oct. 20, 2005

(54) Pin coated assay

(76) Inventors: Jose M. Carballido Herrera, Perchtoldsdorf (AT); Jan E. Vries, Wien (AT); Juliana Kund, Kaltenleutgeben (AT)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/507,254

(22) PCT Filed: May 7, 2003
(86) PCT No.: PCT/EP03/04793

§ 371 Date: Sep. 10, 2004

(30) Foreign Application Priority Data
May 8, 2002 (GB) 0210535.1

(51) Int. Cl. A61K 38/17; G01N 33/53; G01N 33/567; C07K 14/715

(52) U.S. Cl. 514/2; 435/7.2; 530/350

(57) ABSTRACT

A method for identifying an agent that has an influence on the amount of an analyte expressed by a cell, said method comprising stimulating a cell to produce at least 2 analytes, forming, in the absence and in the presence of a candidate compound, on the pins of a matrix at least 2 recognition complexes between an analyte and a recognition molecule, treating said recognition complexes with detection molecules to obtain on the pins of the matrix detection complexes, determining each amount of each detection complex formed on the pins, comparing each amount of detection complex formed in the absence and in the presence of a candidate compound, and choosing a candidate compound which has an influence on the amount of at least one of the detection complexes formed as a pharmaceutical agent.

The present invention relates to a pin coated assay and to a method for detecting multiple analytes expressed by cells in a medium, e.g. a method for identifying an agent that has an influence on the expression of an analyte by a cell.

Generally, assays for detecting such analyte in a medium may be run by contacting a medium containing such analyte with a recognition molecule, and optionally further contacting a recognition complex formed with a detection molecule and determining the amount of detection complex formed. Said contacts may be performed In micro titer plate wells containing such analyte. E.g. in case that several analytes are desired to be determined a complicated procedure may be necessary.

We have now found that such complicated procedure may be facilitated.

In one aspect the present invention provides a method for identifying an agent that has an influence on the amount of an analyte expressed by a cell, said method comprising the following steps
(a) providing a medium comprising a cell with the ability to express at least 2 analytes upon stimulation,
(b) providing means for stimulation of said cell to express such analytes,
(c) providing a candidate compound,
(d) contacting the medium of (a) with the means for stimulation of (b) for a sufficient period of time to obtain a medium comprising a stimulated cell, and adding a candidate compound before, simultaneously or shortly after contacting; or adding no candidate compound,
(e) optionally disrupting cells,
(f) providing a matrix comprising pins which pins are coated with a coating mixture comprising at least 2 different recognition molecules, from each of which it is known that it will bind at a specific binding site to one of the analytes,
(g) contacting the pins of the matrix of (f) with the medium obtained in (d) for a sufficient period of time to allow the formation of recognition complexes on the pins of said matrix, each recognition complex being a complex formed by binding of one single analyte to its specific recognition molecule,
(h) providing at least 2 different detection molecules, from which detection molecules it is known that each will bind to a specific binding site of one of the recognition complexes formed in (g) without interfering with the binding of said analyte to its recognition molecule,
(i) contacting the detection molecules of (h) with the pins of the matrix obtained in (g) for a sufficient period of time to allow the formation of detection complexes on the pins of said matrix, each detection complex being a complex formed by binding of one single recognition complex to its specific detection molecule,
(j) determining each amount of each detection complex formed on the pins in (i),
(k) comparing each amount of detection complex formed in the absence and In the presence of a candidate compound, and
(l) choosing an agent which has an influence on the amount of at least one of the detection complexes formed as determined in (j) and (k).

According to the present invention
a “cell” includes one or more cells or cell lines, preferably one cell line, with the ability to express at least 2 analytes upon stimulation in a medium,
an “analyte” is the material to be assayed,
at least 2 analytes which are soluble in the medium are simultaneously detected using one single pin system,
analytes may be expressed extra- or intracellularly by a cell (cell line) upon sumulation,
at least 2 analytes are assayed, e.g. 2 to 10, preferably 2 to 6, such as 3 to 4.

Analytes according to the present invention include polypeptides/oligopeptides/oligonucleotides which are capable of mediating in vivo events, such as cytokines, chemokines, (cognate) receptors, antibodies and oligonucleotides.

Cytokines are a class of compounds which regulate responses of cells of the immune system, such as B and T lymphocyte cells (“B cells” and “T cells”), natural killer (“NK”) cells, antigen presenting (“APC”) cells. A “cytokine” is a soluble substance released by certain cell populations on contact with an inducer (stimulant) and which acts as an intercelular mediator. The terms “cytokine” and “lymphokine” have become interchangeable. In an attempt to simplify the nomenclature of these compounds, a group of participants at the Second International Lymphokine Workshop held in 1979 proposed the term “interleukin” (IL) to develop a uniform system of nomenclature based on the ability of the proteins to act as communication signals between different populations of leukocytes. Cytokines preferably include interleukines, preferably IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, interferones, preferably IFN-α, IFN-β, IFN-γ, the tumor necrosis factor (TNF molecules), e.g. TNF-α, TNF-β, the transforming growth factor TGF, including the TGF-β superfamily selected from the group of cytokines consisting of the TGF-β family, the inhibin family, the DPP/VG1 family, and the Mullerian Inhibiting Substance family, and the granulocyte-macrophage colony stimulating factor GM-CSF. Cytokines Include cytokine receptors. “Cytokine (superfamily) receptors” are a group of closely related glycoprotein cell surface and soluble receptors that share considerable homology including frequently a WSXWS domain and are generally classified as members of the cytokine receptor superfamily. Members of the superfamily include, but are not limited to, receptors for: IL-2 (α, β and γ chains), IL-3, IL-4, IL-5; IL-6, IL-7, IL-9, granulocyte-macrophage colony-stimulating factor (GMCSF), granulocyte colony-stimulating factor (G-CSF), Leukemia inhibitory factor (LIF); oncostatin M (OSM) and also receptors for prolactin, growth hormone (GH), ciliary neurotrophic factor (CNTF). These receptors are generally expressed as transmembrane proteins but they can also be found in solution in medium samples.

Chemokines, also known as “intercrines” and “SIS cytokines”, comprise a family of small secreted proteins (e.g. 70-100 amino acids and about 8-10 kiloDaltons) which attract and activate leukocytes and thereby aid in the stimulation and regulation of the immune system. The name “chemokine” is derived from chemotactic cytokine, and refers to the ability of such proteins to stimulate chemotaxis of leukocytes. Indeed, chemokines may comprise the main attractants for inflammatory cells into pathological tissues (see generally, Baggiolini et al., Advances in Immunology, 55:97-179 (1994)). Previously identified chemokines generally may exhibit 20-70% amino acid identity to each other and contain four highly-conserved cysteine residues. Based on the relative position of the first two of these cysteine residues, chemokines have been further classified into subfamilies. In the “C—X—C” or “a” subfamily, encoded by genes localized to human chromosome 4, the first two cysteines are separated by one amino acid. In the “C—C” or “β” subfamily, encoded by genes on human chromosome 17, the first two cysteines are adjacent. X-ray crystallography and NMR studies of several chemokines have indicated that, in each family, the first and third cysteines form a disulfide bridge, and the second and fourth cysteines form a second disulfide bridge, strongly influencing the native conformation of these proteins. Families of chemokine proteins are described in more detail in Zlotnik et al., 12 Immunity 121-27 (2000) and Saunders et al., 4 DDT 80-92 (1999).

A cognate receptor for a chemoattractant is a receptor that can interact with the chemoattractant molecule. Cognate, in general, refers to biomolecules that typically interact. for example, a receptor and its ligand. Chemokines include, but are not limited to, chemokine receptors and preferably include the chemokine receptors CXCR1, CXCR2, CXCR3, CXCR4, CXCR5, CXCR6, CCR1, CCR2, CCR3, CCR4, CCR5, CCR6, CCR7, CCR8, CCR9, CCR10, CCR11, XCR1, CX₃CR1, C5a receptor, arachidonate derivative leukotriene B₄ receptor, platelet activating factor receptors, formyl-met-leu-phe receptor, neutrophil activating protein-1 receptor, interleukin 8 receptor, platelet factor 4 receptor, platelet basic protein receptor, melanoma growth stimulating factor/GRO receptor.

Means for stimulating cells to express analytes according to the present invention are known, or may be found, e.g. analogously to known methods, or are as described herein. Stimulation agents include e.g. antibodies, antigens, superantigens and chemical compounds. Methods for disrupting cells are known or may be found, e.g. analogously to known methods. Cell disruption according to the present invention optionally may be carried out, e.g. in the case that an analyte which is assayed (desired to be detected), is expressed intracellularly, e.g. before performing step f).

A matrix comprising pins of the present invention consists of a matrix wherein pins are located, herein also designated as “pin system”. A matrix is preferably a plastic matrix. Pins are preferably plastic pins or (pre-treated) glass pins, preferably plastic pins, with the ability to fix a coating mixture of the present invention on their surfaces, e.g. directly or via a linker. Plastic or (pre-treated) glass material with the ability to fix (stably) a recognition molecule is known or may be provided analogously to a method as conventional. The pins conveniently should all have the same size. The length of the pins should be such, that all pins conveniently can be dipped into wells (e.g. wells of micro-titer plates), each pin in one single distinct well, simultanously. A preferred pin length include 10 mm and more, e.g. 10 mm to 400 mm, preferably 20 to 300 mm, such as 40 to 200 mm. These pins should have an appropriate thickness which allows convenient coating of the pins and which allows convenient dipping of the pins into wells, e.g. 0.2 to 2.0 mm, such as 0.3 to 1.5 mm. The size of that matrix should be such, that it can be conveniently handled, either by hand or by a (robot) machine. The size of that matrix is e.g. dependent on the number of pins which are desired and the well system were they are intended to be inserted. Preferably 96 and more pins, e.g. 96 to 100000 pins may be used, e.g. a convenient number of pins include that number which fits into existent standard microtiter plates; e.g. 96, 384 or 1536, e.g. arranged in a format that fits existent standard microtiter plates; but miniaturization of pin sizes and matrix formats to accommodate standard oligonucleotide arrays is also an option. The pins are located such, that each pin is separated from its neighboring pins. These pins should be regularly located in the matrix. For that a well system should be provided with a number of wells which corresponds to the number of pins and wherein the wells are located such, that the pins of the pin system can be dipped into the well system simultanously, in a way that each pin is dipped distinctly in one well.

According to the present incvention the pins are coated with a coating mixture comprising at least 2 different recognition molecules, e.g. in appropriate medium, from each of said recognition molecule it is known that it will bind at a specific binding site to one of the analytes, thus resulting in the formation of an analyte bound to a recognition molecule, hereinafter designated as “recognition complex”. Appropriate recognition molecules include for example antibodies, (cognate) receptors, e.g. including fragments of antibodies of (cognate) receptors; and other substances, such as low molecular weight compounds from which their ability to bind to an analyte is known. Examples include e.g.
a cytokine, in case that a cytokine receptor is used as an analyte, or
a cytokine receptor, in case that a cytokine is used as an anyalyte,
a oligonucleotide sequence, in case that an oligonucleotide containing a complementary sequence is used as an analyte.

Pin coating may be effected analogously to a method as conventional, and is preferably simply effected by dipping the pins of the pin system in a well system, wherein the wells are filled with a mixture of at least 2 recognition molecules in appropriate medium, to obtain a matrix comprising pins coated with a recognition molecule.

An analyte is bound to its specific recognition molecule when dipping the coated pins into wells containing such analyte, e.g. by dipping the pins into wells containing stimulated intact cells or by dipping the pins into wells containing stimulated cells after disrupting their membranes to allow the release of intracellular analytes in the medium containing the cells and recognition complexes are formed on the pins. In the absence of a candidate compound a specific amount of such recognition complexes will be formed on the pins, in the presence of a candidate compound which has an influence on the analyte expression, an amount of at least one of the recognition complexes will be formed which is higher or lower than the specific amount formed in its absence. A candidate compound for which such influence is found according to the present invention is designated hereinafter as an (ant)agonist or an agent (according to) the present invention.

A medium comprising a stimulated cell (and therefore analytes) is preferably incubated in the presence of pins coated according to the present for a period of time which is sufficient for the formation of the recognition complexes. A necessary time period may be determined by pre-testing. Contacting according to step g) is preferably carried out by dipping the pins of the pin system as provided according tostep f) in a corresponding well system wherein the wells are filled with a medium comprising a coating mixture according to the present invention.

Formation and amount of such recognition complexes formed may be determined by treating such recognition complex with a detection molecule, preferably with a mixture of at least two detection molecules, from each of which it is known that it will bind to a specific binding site of the analyte in one single recognition complex without interfering with the binding of the analyte to the recognition molecule, thus forming a “detection complex” on the pins of the matrix of the present invention, and determining each amount of each detection complex formed. For the formation of a detection complex the binding site in an analyte for the recognition molecule must be different from the binding site of the detection molecule to said analyte, and the binding of the detection molecule to said analyte in the recognition complex must not interfere with the binding of said analyte to the recognition molecule, i.e. each detection complex is formed in an amount which is that of the correponding recognition complex, i.e. said detection molecule recognizing an epitope of one of the analytes which is different to that recognized by the recognition molecule. Appropriate systems may be found or are known, e.g. as “Sandwich Elisa”-systems, e.g. comprising an “(antibody)(analyte)-(labeled-antibody)-sandwich”.

Contacting coated pins according to step i) of the present invention with a detection molecule may be carried out by dipping the pin system of step g) into at least two well systems wherein the wells are filled in each case with one appropriate detection molecule, or by dipping into a mixture of at least two different appropriate detection molecules. Detection complexes are formed on the pins of the matrix.

A detection molecule includes e.g. an enzyme or a labeled molecule, which labeling may be measured, e.g. quantitatively, e.g. a fluorescence or luminescence labeled molecule. Appropriate labels are known or may be found, e.g. analaogously to a method as conventional. A detection molecule according to the present invention preferably includes horseradish peroxidase substrates, alkaline phosphatase substrates, luciferase substrates, polymerase chain reaction solutions, time resolve fluorescence substrates, such as lanthanides, and optionally enhancement solutions. For labeling preferably lanthanides, e.g. Europium, Terbium, Samarium and Dysprosium are used. Preferred examples for appropriate detection molecules or a mixture of detection molecules include an antibody to human IL-4 labeled with Europium, an antibody to human IFN-γ labeled with Samarium, an antibody to human IL-10 labeled with Terbium, or individual mixtures of such labeled antibodies.

Determination of the amount of analyte formed according to step j) may be carried out by isolating such detection complex, i.e. by simply removing the matrix system from the well system, and measuring the effect of the detection molecule formed on the pins of the matrix system. Such measurement may be done by use of means for detecting the amount of a detection complex formed according to the present invention in normal assays, e.g. ELISA, DELFIA and oligonucleotide tagging assays. Preferably the amount of each analyte on the pins is detected by dipping the pin system in a well system wherein the wells are filled with an appropriate detection element, e.g. in appropriate medium. An appropriate detection medium includes e.g. a substrate medium containing a detection molecule that is capable to change optical or fluorescence properties in contact with the coating on the pins by forming detection complexes. Appropriate detection molecules are known or may be found according, e.g. anlogously, to a method as convential and e.g. include horseradish peroxidase substrates, alkaline phosphatase substrates, luciferase substrates, time resolve fluorescence substrates, e.g. using lanthanide-labels, and enhancement solutions, and polymerase chain reaction solutions., e.g. in case of an enzyme as a detection molecule, measuring the enzymatic actity, or, in case of a labeled reagent, measuring a label-specific effect, e.g. in case of fluoresence-abeling, measuring the label-specific effect by appropriate luminescence/fluorescence determination methods at appropriate wavelengths, e.g. including methods as conventional.

A candidate compound according to the present invention includes low molecular weight compounds, e.g. in chemical libraries and natural product libraries, and antisense oligonucleotides, e.g. present in natural or synthetic compound libraries, i.e. systematic collections of chemical entries, for which the effect on the production of analyte-expression in a cell according to step (b) is unknown and desired to be assayed.

An (ant)agonist(s) Identified according to a method of the present invention, herein also designated as “agent(s) of (according to) the present invention” is one of the chosen candidate compounds for which an influence on the amount of an analyte expressed by a cell according to the present invention has been determined. An agent may be an agonist or an antagonist, e.g. Including (oligo- or poly-)peptides, monoclonal antibodies, chemical compounds, such as low molecular compounds, natural occurring compounds, antisense oligonucleotides. (Ant)agonists identified according to the present invention, because of their ability to interfere in the production of an analyte in vivo, may be useful as pharmaceutically active compounds and/or as diagnostic tools.

A method according to the present invention may be advantageous in comparison with prior art processes because at least 2 different analytes may be determined with one single pin system which is simply dipped into wells comprising
a medium comprising cells expressing at least two analytes upon stimulation,
a medium comprising a mixture of detection molecules,
   in the absence and in the presence of candidate compounds.

Thus, one single pin matrix is used to determine at least two, or even more, different analytes simultaneously. The determination process may be repeated, e.g. in the same wells comprising the analytes but using a pin system wherein the pins are coated with a different mixture of recognition molecules compared with the pin system in the first run. This may double the number of analytes which may be determined. Washing procedures between distinct determination steps, such as between contacting with recognition molecule and contacting with a detection element, e.g. as necessary in ELISA detection procedures, may be avoided or simplified, e.g. comprising dipping the pin matrix system simply in wells comprising a washing medium, if washing is desired.

In another aspect the present Invention provides a kit for identifying an agent that has an influence on the amount of an analyte expressed by a cell, which kit comprises
(a) a medium comprising a cell with the ability to express at least 2 analytes upon stimulation,
(b) means for stimulating said cell to express such analytes,
(c) optionally means for cell disruption,
(d) a matrix comprising pins which are coated with a coating mixture comprising at least 2 different recognition molecules, from each of which it is known that it will bind at a specific binding site to one of the analytes, thus forming a recognition complex upon contact with one of the analytes of (a) on the pins,
(e) at least 2 detection molecules, from which detection molecules it is known that each will bind to a specific binding site of one of the recognition complexes formed according to (d) without interfering with the binding of said analyte to its recognition molecule, thus forming a detection complex upon contact with a recognition complex formed according to (d) on the pins,
(f) means for determining the amount of a detection complex formed on said pins.
(g) well system(s) wherein the number and form correspond to the pins comprised in the matrix of (d),
(h) optionally calibration standards for analytes expressed by cells of (a), e.g. calibration sample(s),
(i) optionally control sample(s) containing known amounts/concentrations of analytes expressed by cells of (a), and
(j) optionally instructions for using the components of said kit to quantify or to detect analytes expressed by cells of (a) in a sample.

In a kit of the present invention any component (a) to (i) may comprise a substantial component, e.g. including an appropriate environment for a sample to be tested. A kit according to the present invention may also be useful as a diagnostic kit.

In another aspect the present invention provides a kit according to the present invention for use as a diagnostic kit, e.g. in autoimmune related diseases or in allergic diseases.

Means for detecting the amount of the detection complex formed on the pins of the pin system include means as indicated above. Calibration standards for analytes expressed by cells of (a) and control sample(s) may be provided as appropriate, e.g. according, e.g. analogously, to a method as conventional. CD4⁺ T helper cells (Th cells) play a major role in regulating the effector mechanisms that the adaptive immune system has developed to combate different pathogens. Th cells recognize specific antigen peptides, presented by professional antigen presenting cells (APC), in the context of Major Histocompatibility Class II (MHC-II) molecules. Upon activation, Th cells proliferate and differentiate into discrete Th cell-subsets defined on the basis of their cytokine secretion patterns. Th cell differentiation is a multifactorial decision determined by factors such as the genetic background, the dose of antigen, the density of costimulatory molecules and, above all, the cytokine milieu in which the immune response takes place (see e.g. Coffman R L and Reiner S L, Science (1999), 284: 1283). Thus, IL-12 is responsible for driving Th cells into the Th1 phenotype, which is characterized by the production of IL-2, IFN-γ and TNF-β. Th1 cells are involved e.g. in pro-inflammatory responses that are characteristic of cell-mediated immunity and therefore they are important for the clearance of many infectious organisms. Th2 cells e.g. produce IL-4, IL-5 and IL-13 and their development is induced by IL-4. Th2 cells are predominantly helper of antibody-mediated B cell responses and exert anti-inflammatory functions. Generally, naive CD45RA⁺ Th cells first progress to a Th0 phenotype, a subset that is capable of producing both Th1 and Th2 cytokines. Following repeated stimulation with specific antigens, Th0 cells differentiate further into Th1 and Th2 cells. The resulting Th1 or Th2 cell subsets remain predominant throughout a particular immune episode in part because of counter-inhibitory effects of cytokines secreted by the reciprocal subset (see e.g. O'Garra A. Immunity (1998), 8: 275). However, exacerbated Th1 or Th2 responses might be detrimental for the host. Hence, Th1 cells are implicated in the immunopathology associated with autoimmune diseases whereas Th2 cells mediate allergic disorders. Fortunately, most human Th cells co-express various combinations of Th1 and Th2 cytokines. These Th0 cells may also represent stable subsets with balanced Th1/Th2 cytokine production that are functionally involved in the clearance of pathogens while inducing minimal immunopathology. In addition, CD4⁺ T cells may also differentiate, in the presence of IL-1 0, into T regulatory (Tr1)-cells. Tr1-cells produce high levels of IL-10 and TGF-β1 and exert downregulatory effects on both Th1 and Th2 responses (see e.g. Groux H, O'Garra A, Bigler M, Rouleau M, Antonenko S, De Vries J E and Roncarolo M G, Nature (1997), 389: 737).

It is evident that the immune system has evolved to generate the type of immune response most appropriate for each particular situation and that, in addition, it has developed control mechanisms to switch off these responses once the danger signals are cleared. However, despite of these regulatory mechanisms, situations of extreme Th polarization resulting in immunopathology often occur and then, it would be extremely beneficial to have a suitable active substance capable of antagonizing the effector functions of the polarizing cytokine and/or reverting the ongoing immune response into a non-pathological outcome. According to the present invention we also have found the basis of a high throughput screening (HTS) assay to identify modulators of human IL-4, IFN-γ and IL-10 production. These cytokines play Important roles in the processes of differentiation and/or effector functions of Th cells, including immunopathological situations. Antagonists, e.g. specific antagonists, of IL-4 production would be candidates to target allergic diseases, antagonists, e.g. specific antagonists, supressing IFN-γ production may be beneficial for the treatment of autoimmune diseases, and agonists, e.g. specific agonists, of IL-10 production would be potential downregulators of immune responses and are useful in the treatment and prophylaxis of corresponding diseases.

In another aspect the present invention provides a method for identifying an agent that has an influence on the amount of an analyte expressed by a cell according to the present invention, wherein
the analytes are selected from the group consisting of human IL-4 and/or IL-10 and/or IFN-γ,
the recognition molecule is selected from the group consisting of antibodies to human IL-4, IL-10 and IFN-γ.
said detection molecule is selected from the group consisting of labeled antibodies to human IL-4, IL-10 and IFN-γ, said detection molecule recognizing an epitope of said analyte which is different to that recognized by the recognition molecule, e.g. wherein said antibodies are labeled by a lanthanide, such as Europium, Terbium, Samarium and Dysprosium.

A specific pharmaceutical activity of an agent of the present invention, e.g. an (ant)agonist, is dependent e.g. on the bological activity of the analytes detected and includes pharmaceutial activity in diseases which are mediated by the release of specific analyte(s) in vivo, e.g. wherein analyte(s) are selected from proteins which are capable of mediating in vivo events, such as cytokines, chemokines, (cognate) receptors, antibodies and oligonucleotides.

An agent of the present invention may show therapeutic activity against autoimmune related diseases and/or allergic diseases.

In another aspect the present invention provides
an agent of the present invention for use as a pharmaceutical,
an agent of the present invention for the manufacture of a medicament for the treatment of autoimmune related diseases or allergic diseases.

An agent of the present invention for treatment includes one or more, e.g. a combination of two or more, preferably one, agent of the present invention.

In another aspect the present invention provides a pharmaceutical composition comprising an agent of the present invention in association with at least one pharmaceutical excipient, e.g. appropriate carrier and/or diluent, e.g. including fillers, binders, disintegrators, flow conditioners, lubricants, sugars and sweeteners, fragrances, preservatives, stabilizers, wetting agents and/or emulsifiers, solubilizers, emollients, moisterizing agents, salts for regulating osmotic pressure and/or buffers.

Such compositions may be manufactured according, e.g. analogously, to a method as conventional, e.g. by mixing, granulating, coating, dissolving or lyophilizing processes. Unit dosage forms may contain, for example, from about 0.5 mg to about 1000 mg, such as 1 mg to about 500 mg.

In a further aspect the present invention provides a method of treatment of autoimmune related diseases or allergic diseases, which treatment comprises administering to a subject in need of such treatment an effective amount of an agent of the present invention, e.g. or of a pharmaceutical composition of the present invention.

Treatment includes treatment and prophylaxis.

For such treatment, the appropriate dosage will, of course, vary depending upon, for example, the chemical nature and the pharmacokinetic data of an agent of the present invention employed, the individual host, the mode of administration and the nature and severity of the conditions being treated. However, in general, for satisfactory results in larger mammals, for example humans, an indicated daily dosage is in the range from about 0.01 g to about 1.0 g, of an agent of the present invention; conveniently administered, for example, in divided doses up to four times a day.

An agent of the present invention may be administered by any conventional route, for example enterally, e.g. including nasal, buccal, rectal, oral, administration; parenterally, e.g. including intravenous, intramuscular, subcutanous administration; or topically; e.g. including epicutaneous, intranasal, intratracheal administration; e.g. in form of coated or uncoated tablets, capsules, injectable solutions or suspensions, e.g. in the form of ampoules, vials, in the form of creams, gels, pastes, inhaler powder, foams, tinctures, lip sticks, drops, sprays, or in the form of suppositories.

An agent of the present invention may be administered in the form of a pharmaceutically acceptable salt, e.g. an acid addition salt or metal salt; or in free form; optionally in the form of a solvate. An agent of the present invention in the form of a salt may exhibit the same order of activity as that agent in free form; optionally in the form of a solvate.

An agent of the present invention may be used for pharmaceutical treatment according to the present invention alone, or in combination with one or more other pharmaceutically active agents. Combinations include fixed combinations, in which two or more pharmaceutically active agents are in the same formulation; kits, in which two or more pharmaceutically active agents in separate formulations are sold in the same package, e.g. with instruction for co-administration; and free combinations in which the pharmaceutically active agents are packaged separately, but instruction for simultaneous or sequential administration are given.

In another aspect the present invention provides the use of an agent of the present invention as a diagnostic/surrogate molecule.

### DESCRIPTION OF THE FIGURES

FIG. 1 shows cytokine production of #98016T0 cells. T cell stimulation is performed as described under Preparation of a cell line which produces analyte-containing medium below, at day 12 after re-stimulation. Intracytoplasmic staining is performed after 4 hours of activation to determine the cytokine production profile at the single cell level. The total amount of cytokine production measured by ELISA after 24 hours of activation is 5.2 ng/ml of IL-2, 13.7 ng/ml of IFN-γ, 7.9 ng/ml of IL-4, 0.1 ng/ml of IL-5 and 24.6 ng/ml of IL-10 and correlated with the phenotype determined by intracytoplasmic staining.

FIG. 2 shows the detection of recombinant human cytokine standards following the DELFIA procedure. Recombinant human IL-4 (R&D Systems, Minneapolis, Minn.), IL-10 (PharMingen) and IFN-γ (R&D Systems) are used as standards. Washing and blocking solutions are the same as for the ELISA method as described above. All samples are assayed in triplicate. Each point represents the average result±standard deviation. All 3 cytokiners are detected in a mix solution using issue culture supernatant as diluent These results indicate that the triple-DELFIA detects <50 pg/ml of each cytokine.

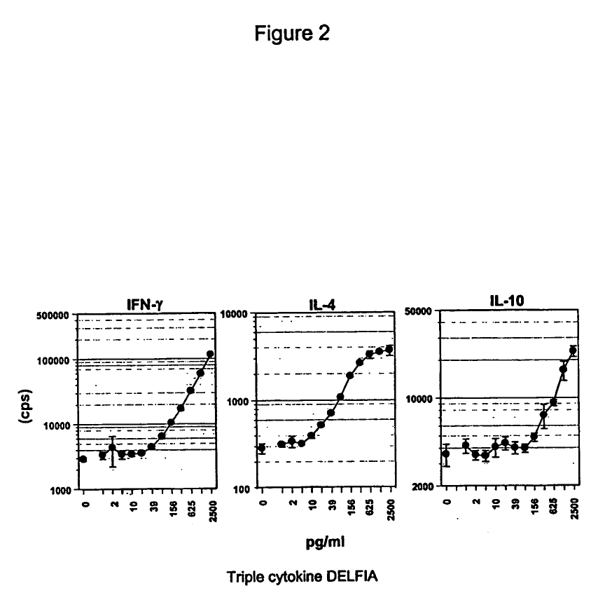

FIG. 3 shows results obtained using T-cell supernatants of serial dilutions of stimulated #98016To cells. These results are obtained using anti-CD3 immunobeads plus anti-CD28 mAb as stimulatory signals (filled circles). The cytokine levels present In the supematants of unstimulated cells (emty circles) do not differ significantly from the levels observed in the wells containing medium alone (without cells). Both, T cell stimulation and DELFIA assays are carried out using 384-well plates as described below. All samples are assayed in triplicate. Each point represents the average result±standard deviation.

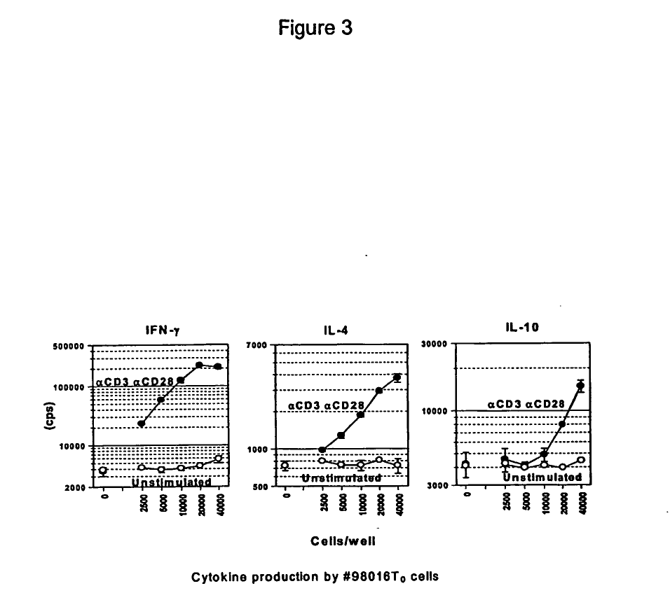

FIG. 4 illustrates a characteristic dose dependent inhibition of IFN-γ, IL-4 and IL-10 production of #98016T0 cells stimulated in the presence of increasing amounts of the immunosuppressant Cyclosporin A (CsA) in an assay according to the present invention. T cells are activated for 24 hours with anti-CD3 immunobeads and anti-CD28. All samples are assayed in triplicate. Each point represents the average result±standard deviation.

In the following examples all temperatures are given in degree Celcius and are uncorrected.

### EXAMPLES

### Example 1

Preparation of a Cell Line Which Produces Analyte-containing Medium

IL-4, IFN-γ and IL-10 are chosen as analytes.

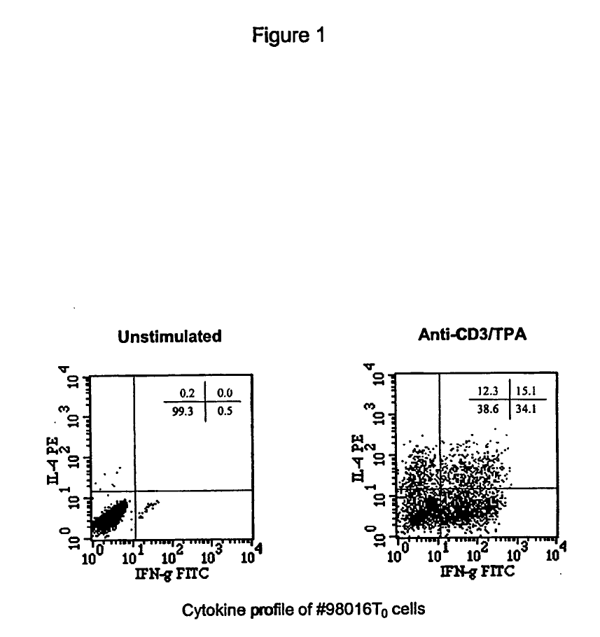

A human CD4⁺ Th₀ cell line, i.e. cell line #98016T₀, from a skin biopsy of a patient with atopic dermatitis (AD) is isolated according to the method as described in Carballido J M, Aversa G. Kaltoft K, Cocks B G, Punnonen J, Yssel H. Thestrup-Pedersen K and de Vries J E. J Immunol (1997), 159: 4316. Briefly, patients with AD, who are allergic to Der p 1 as judged by specific cutaneous prick test and serum IgE levels are patch challenged with Der p 1. Punch biopsies (4 mm) are obtained from the patients that underwent a positive skin reaction after 24 hours exposure to the allergen, cut In four approximately equally sized pieces and cultured at 37° in a humidified atmosphere under 8% CO₂ in 12-well plates (Costar, Cambridge, Mass.) in Yssel's medium (Gibco BRL) containing 1% human AB serum and 1 μg/ml of purified Der p 1 (ALK Laboratories, Horsholm, Denmark). The cultures are supplemented with 100 U/ml of rhIL-2 and 400 U/ml of rhIL-4 in the presence and in the absence of 0.3 ng/ml of rhIL-12 (R&D Systems, Minneapolis, Minn.). Th cells migrate out of the skin biopsies and proliferated with comparable doubling times under the different culture conditions. Culture medium containing the relevant cytokines is replaced every 2 to 3 days. After 14 days of expansion, the cell cultures are re-stimulated with 1 μg/ml of purified Der p 1 and re-expanded in medium supplemented with the cytokine cocktails. At the end of the second culture cycle, >5×10⁷ cells are recovered from each condition. Subsequently, human Th cell lines are maintained in Yssel's medium 10% FCS by repeated stimulation using 1 μg/ml PHA and allogeneic (50 Gy irradiated) peripheral blood mononuclear cells, as a source of feeder cells, followed by expansion on IL-2 and IL-4 i IL-12. The CD4⁺ cell line #98016T₀ is selected because these cells, upon stimulation, consistently displayed a Th₀ phenotype as judged by intracytoplasmic cytokine staining detected at the single cell level by FACS or by cytokine production at the total population level measured by ELISA (see e.g. also FIG. 1).

FACS and ELISA (Proof of Cytokine Production by the Selected Cell Line)

Human #98016T₀ cells are harvested 12 to 16 days after re-stimulation, washed three times in PBS and stimulated in 24-well plates (Costar) using Yssel's medium containing 10% FCS, 5 μg/ml of the anti-CD3 mAb SPV-T3(5) and 1 ng/ml of 12-O-tetradecanoylphorbol-13-acetate (TPA) (SIGMA, St Louis, Mo.). The intracytoplasmic content of IL-4 and IFN-γ in single cells is determined according to the method as described in Carballido J M, Aversa G. Kaltoft K, Cocks B G, Punnonen J, Yssel H, Thestrup-Pedersen K and de Vries J E. J Immunol (1997), 159: 4316. Briefly, the Th cells are treated with 10 μg/ml of Brefeldin-A (Epicentre Technologies, Madison, Wis.) 2 hours after stimulation and cultured for additional 2 hours. Next, the Th cells are harvested, fixed in 2% formaldehyde, permeabilized with saponin and stained with anti-IL-4 and anti-IFN-γ mAb conjugated to PE and FITC, respectively (PharMingen, San Diego, Calif.). The Th cells are analyzed using a FACScan flow cytometer and CellQuest software (Becton Dickinson, San Jose, Calif.). To determine total cytokine production the Th cells are harvested and stimulated for 24 h at 10⁶ cells/well using 24-well plates (Costar) In 1 ml of Yssel's medium containing 10% FCS, 5 μg/ml of the anti-CD3 mAb SPV-T3 and 1 ng/ml of TPA. Cytokine production is measured by specific sandwich ELISA as follows: Maxisorp C96-well immuno-plates (NUNC, Roskilde, Denmark) are coated overnight at room temperature with 50 μl/well of 3 μg/ml solution of specific capturing mAb in Carbonate buffer (SIGMA C-3041). After coating, the plates are washed with PBS-0.05% Tween 20 and blocked at room temperature by 1 hour incubation with 200 μl of casein hydrolysate (OXOID L41) and 5% Tween 20 in PBS. Thereafter, the plates are incubated with 25 μ/well of the different T cell supernatants or specific standard plus 25 μl/well of biotinylated mAb. After overnight incubation at room temperature the plates are washed again and incubated for 2 hours with extravidin alkaline phosphatase conjugated (SIGMA E-2636). Read out is performed using pnitrophenyl phosphate (SIGMA N-2640) in 1 M diethanolamine buffer as a substrate and measuring absorbance at 405-492 nm in a SpectraMAX 340 spectrophotometer (Molecular Devices, Sunnyvale, Calif.) equipped with SoftMax software (Molecular Devices). The mAbs used for capturing and detection, respectively, are 8F12 and 3H-4 for the IL-4 ELISA and 43-11 and 45-15 for the IFN-γ ELISA. Production amounts of some cytokines are shown in FIG. 1.

### Example 2

HTS Assay for Human Cytokine Inhibitors

Step 1. T Cell Stimulation

Anti-CD3 mAb SPV-T3 is coupled to 100 mg of activated immunobead matrix (Irvine Scientific, Santa Ana, Calif.) according to the manufacturer's instructions. The ant-CD3 coupled immunobeads are titrated in T-cell proliferation assays and used in all successive experiments at the final concentration of 75 μg/ml (equivalent to 2 μg/ml of anti-CD3 mAb). Anti-CD28 mAb (provided by PharMingen, San Diego, Calif.) is used at 5 μg/ml. Serial dilutions of #98016T₀ cells are stimulated for 24 hours with anti-CD3 immunobeads and anti-CD28 mAb in Yssel's medium in the presence or absence of a candidate compounds. T-cell stimulation is performed in 40 μl volume using 384-well tissue culture plates (Nunc #164688).

Step 2. Pin Coating with Recognition Molecules and Complex Formation

As recognition molecules commonly used mAbs against IL-4, IL-10 and IFN-γ are used. Coating mAbs for IL-4, IL-10 and IFN-γ DELFIA are designated herein as 8F12, JES3-12G8 and 43-11, respectively. Cytokine production is determined by time-resolved fluorescence in a Wallac 1420 Victor2 multilabel counter.

For the purpose of pin coating, the pins of a matrix wherein 384 pins are located, which pins fit into a 384-well tissue culture plate (Nunc #164688) are coated by dipping these pins for 2 hours at room temperature into 30 μl/well of a cocktail containing 2 μg/ml of mAb 8F12, 2.5 μg/ml of mAb JES3-12G8 and 2.5 μg/ml of mAb 43-11 in carbonate buffer, pH 9.6 (SIGMA C3041) and blocking for 1 hour at room temperature. Blocking is performed by dipping the mAb-coated pins in a 384-well plate containing a solution of casein hydrolysate (OXOID L41) and Tween-20 in PBS.

Step 3. Contact With Detection Molecules and Determination of Analyte Content

For determination of detection complex formation on the pins Enhanced Lanthanide FluoroImmuno Assay (DELFIA) is used.

As detection molecules mAbs for IL-4, IL-10 and IFN-γ DELFIA are used which do not compete with the recognition molecule mAbs described in Step 2 above, designated as 3H4 (for IL-4), JES3-9D7 (for IL-10) and 45-15 (for IFN-γ), respectively. The 3H4 mAb is labeled with Samarium (Sm), the JES3-9D7 mAb is labeled with Terbium (Tb) and the 45-15 mAb is labeled with Europium (Eu). Lanthanide labeled mAbs may be custom-labelled by Advant-Wallac, Turku, Finland. Recombinant human IL-4 (R&D Systems, Minneapolis, Minn.), IL-10 (PharMingen) and IFN-γ (R&D Systems) are used as standards. Washing and blocking solutions are the same as for the ELISA method described above. DELFIA assay buffer #1244-111, enhancement solution #1244-105 and enhancer #C500-100 are obtained from Wallac.

The capturing of the specific analytes by the coated pin system (formation of recognition complexes) is achieved by incubation of the pins obtained in step 2 into the 384-well tissue culture plate prepared as described in step 1 (activation of T cells for 24 hours with stimulation agents and candidate compounds). The pin system obtained is dipped into a new 384 well plate in which each well is filled with 30 μl/well of a solution containing the three lanthanide-labeled mAbs (each at 50 μg/ml final) in DELFIA assay buffer, followed by dipping these pins into a 384-well tissue culture plate (Nunc #164688) in which each well Is filled with 50 μl/well of Wallac Enhancer Solution. This last 384-well tissue culture plate (Nunc #164688) in which the pin matrix is immersed is transferred to the reader of time resolved fluorescence determination of Eu and Sm windows and the Eu(IFN-γ)- and Sm(IL-4) content is determined. Thereafter each well of this 384 plate receives additional 15 μl of Wallac Enhancer before immersing again the pin matrix. Finally the plate is read in the Th window to determine the Tb (IL-10)-content.

### Example 3

Inhibition of Cytokine Production by a Known Cytokine-production Inhibitor Determined in an HTS Assay for Human Cytokine Inhibitors

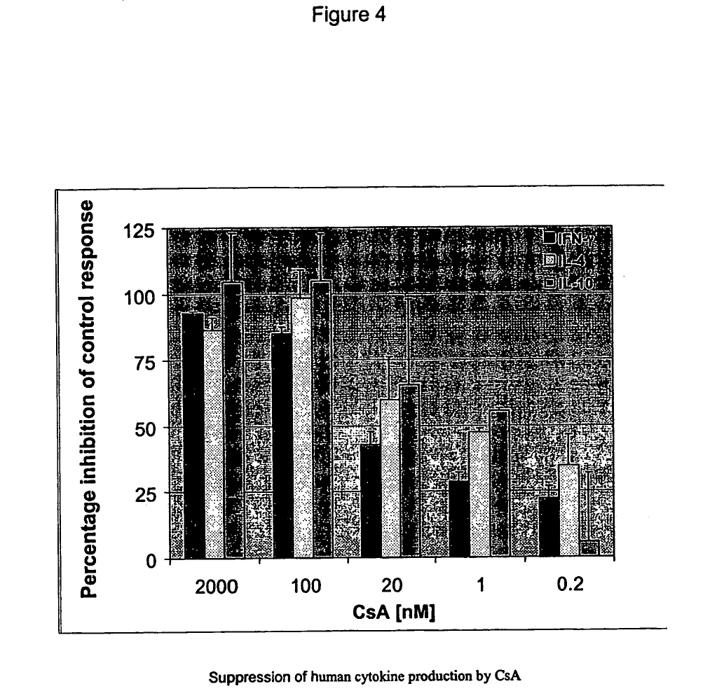

Analaogously to the methods of Example 2, Steps 1 to 3 and using Cyclosporin A (CsA) as a prototype inhibitor (candidate compound) of cytokine production experiments for a proof of principle are carried out. For that, T-cell stimulation according to Example 2, Step 1 is carried out in the presence of different amounts of CsA or without the presence of CsA and the other Steps and determination of cytokine production are carried out according to Example 2, Step 2 and Step 3. Results are shown in FIG. 4 which illustrates a characteristic dose dependent inhibition of IFN-γ, IL-4 and IL-10 production of #98016T0 cells by CsA. This illustrates also the proof of principle of the present invention.

1. A method for identifying an agent that has an influence on the amount of an analyte expressed by a cell, said method comprising the following steps
(a) providing a medium comprising a cell with the ability to express at least 2 analytes upon stimulation,
(b) providing means for stimulation of said cell to express such analytes,
(c) providing a candidate compound,
(d) contacting the medium of (a) with the means for stimulation of (b) for a sufficient period of time to obtain a medium comprising a stimulated cell, and adding a candidate compound before, simultaneously or shortly after contacting; or adding no candidate compound,
(e) optionally disrupting cells,
(f) providing a matrix comprising pins which pins are coated with a coating mixture comprising at least 2 different recognition molecules, from each of which it is known that it will bind at a specific binding site to one of the analytes,
(g) contacting the pins of the matrix of (f) with the medium obtained in (d) for a sufficient period of time to allow the formation of recognition complexes on the pins of said matrix, each recognition complex being a complex formed by binding of one single analyte to its specific recognition molecule,
(h) providing at least 2 different detection molecules, from which detection molecules it is known that each will bind to a specific binding site of one of the recognition complexes formed in (g) without interfering with the binding of said analyte to its recognition molecule,
(i) contacting the detection molecules of (h) with the pins of the matrix obtained in (g) for a sufficient period of time to allow the formation of detection complexes on the pins of said matrix, each detection complex being a complex formed by binding of one single recognition complex to its specific detection molecule,
(j) determining each amount of each detection complex formed on the pins in (i),
(k) comparing each amount of detection complex formed in the absence and in the presence of a candidate compound, and
(l) choosing an agent which has an influence on the amount of at least one of the detection complexes formed as determined in (j) and (k).

2. The method of claim 1 wherein said analyte is selected from the group consisting of human IL-4, IL-10 and IFN-γ.

3. The method of claim 1, wherein said recognition molecule is selected from the group consisting of antibodies to human IL-4, IL-10 and IFN-γ.

4. The method of claim 1 wherein the said detection molecule is selected from the group consisting of labeled antibodies to human IL-4, IL-10 and IFN-γ, said detection molecule recognizing an epitope of said analyte which is different to that recognized by the recognition molecule.

5. A kit for identifying an agent that has an influence on the amount of an analyte expressed by a cell, which kit comprises
(a) a medium comprising a cell with the ability to express at least 2 analytes upon stimulation,
(b) means for stimulating said cell to express such analytes,
(c) optionally means for cell disruption,
(d) a matrix comprising pins which are coated with a coating mixture comprising at least 2 different recognition molecules, from each of which it is known that it will bind at a specific binding site to one of the analytes, thus forming a recognition complex upon contact with one of the analytes of (a) on the pins,
(e) at least 2 detection molecules, from which detection molecules it is known that each will bind to a specific binding site of one of the recognition complexes formed according to (d) without interfering with the binding of said analyte to its recognition molecule, thus forming a detection complex upon contact with a recognition complex formed according to (d) on the pins,
(f) means for determining the amount of a detection complex formed on said pins.
(g) well system(s) wherein the number and form correspond to the pins comprised in the matrix of (d),
(h) optionally calibration standards for analytes expressed by cells of (a),
(i) optionally control sample(s) containing known amounts/concentrations of analytes expressed by cells of (a), and
(j) optionally instructions for using the components of said kit to quantify or to detect analytes expressed by cells of (a) in a sample.

6. An agent identified by a method according to claim 1.

7. An agent of claim 6 for use as a pharmaceutical.

8. An agent identified by a method according to claim 2 for the manufacture of a medicament for the treatment of autoimmune related diseases or allergic diseases.

9. A pharmaceutical composition comprising an agent identified by a method according to claim 1 in association with at least one pharmaceutical excipient.

10. A method of treatment of autoimmune related diseases or allergic diseases, which treatment comprises administering to a subject in need of such treatment an effective amount of an agent identified according to claim 2.
